(19) United States
(12) Patent Application Publication
(10) Pub. No.: US 2003/0152541 A1
(43) Pub. Date: Aug. 14, 2003

(54) Oxidizing composition for treating keratin fibres, comprising a particular aminosilicone

(76) Inventors: Frederic Legrand, Courbevoie (FR); Jean-Marie Millequant, Saint-Maur des Fosses (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/290,342

(22) Filed: Nov. 8, 2002

(30) Foreign Application Priority Data
Nov. 8, 2001 (FR) 01 14472

(51) Int. Cl. A61K 7/021; A61K 7/06; A61K 7/11

(52) U.S. Cl. 424/70.12

(57) ABSTRACT

A cosmetic composition for treating human keratin fibres, such as hair, comprising, in a cosmetically acceptable medium:

(i) at least one oxidizing agent, and

(ii) at least one amino silicone comprising at least one aminoethylimino(C₄-C₈)alkyl group, and its uses for dyeing, bleaching and permanently reshaping human keratin fibres; as well as the processes for bleaching, dyeing or permanently reshaping human keratin fibres using the composition.

This disclosure relates to a novel oxidizing composition for treating human keratin fibres, such as hair, comprising, at least one aminosilicone comprising at least one aminoethylimino(C₄-C₈)alkyl group. This disclosure also relates to uses of the composition for dyeing, permanently reshaping and/or bleaching the human keratin fibres.

It is a well known practice to bleach keratin fibres, for example, human keratin fibres such as hair, with bleaching compositions comprising one or more oxidizing agents. Among the oxidizing agents conventionally used, mention may be made of hydrogen peroxide or compounds capable of producing hydrogen peroxide by hydrolysis, such as urea peroxide or persalts such as perborates, percarbonates and persulphates.

Such bleaching compositions are mainly in the form of anhydrous products (powders or creams) comprising alkaline compounds (amines and alkaline silicates), and a peroxygenated reagent such as ammonium or alkali metal persulphates, perborates or percarbonates, which is diluted at the time of use with an aqueous hydrogen peroxide composition.

The bleaching compositions may also result from the mixing, at the time of use, of the anhydrous peroxygenated reagent powder with an aqueous composition comprising alkaline compounds and another aqueous composition comprising hydrogen peroxide.

The bleaching compositions may also be in the form of ready-to-use thickened aqueous hydrogen peroxide compositions.

As used herein, the expression “ready-to-use composition” means the composition intended to be applied in unmodified form to the keratin fibres, i.e., it may be stored in unmodified form before use or may result from the extemporaneous mixing of two or more compositions.

It is moreover known practice to dye human keratin fibres, such as human hair, with dye compositions comprising oxidation dye precursors, such as ortho- and para-phenylenediamines, ortho- and para-aminophenols, and heterocyclic compounds, generally known as oxidation bases. Oxidation dye precursors or oxidation bases, are colorless or weakly colored compounds, that, when combined with oxidizing products, may give rise to colored compounds and colorants by a process of oxidative condensation. It is also well known that the shades obtained with these oxidation bases may be varied by combining them with couplers or coloration modifiers. These couplers or coloration modifiers may be chosen from, for example, aromatic meta-diamines, meta-aminophenols, meta-diphenols and certain heterocyclic compounds such as indole compounds.

The oxidizing agent present in the oxidizing composition as defined above may be chosen from the oxidizing agents conventionally used for the oxidation dyeing of keratin fibres. The oxidizing agent may be chosen from, for example, hydrogen peroxide and compounds capable of producing hydrogen peroxide by hydrolysis, such as urea peroxide, and persalts such as perborates and persulphates.

It is well known that the most common technique for obtaining a permanent reshaping of the hair comprises, in a first stage, in opening the keratin —S—S-disulphide (cystine) bonds using a composition comprising a suitable reducing agent (reduction step) followed, after having rinsed the head of hair thus treated, by reconstituting, in a second stage, the said disulphide bonds by applying to the hair, which has been placed under tension beforehand (curlers and the like), an oxidizing composition (oxidation step, also known as the fixing step) so as finally to give to the hair the desired shape. This technique thus makes it equally possible either to make the hair wavy or to straighten it and remove its curliness. The new shape given to the hair by a chemical treatment such as above can be remarkably long-lasting and, for example, can withstand the action of washing with water or shampoos, as opposed to simple standard techniques for temporary reshaping, such as hairsetting.

The reducing compositions that may be used for the first step of a permanent-waving operation comprise, as reducing agents, sulphites, bisulphites, alkylphosphines and, thiols.

The oxidizing compositions for carrying out the fixing step can usually be compositions based on aqueous hydrogen peroxide solution.

Moreover, it is well known that the oxidizing treatments in the permanent reshaping of the hair, including those involved in dyeing and bleaching, can often be aggressive and lead to poor cosmetic properties of the hair such as difficulty in disentangling, an unpleasant feel, or coarse, dull hair, or alternatively, hair charged with static electricity, and to the degradation of the fibres.

After considerable research, the inventors have discovered that by using at least one aminosilicone comprising at least one aminoethylimino(C₄-C₈)alkyl group, such as defined below, in an oxidizing composition for permanently reshaping, dyeing and/or bleaching human keratin fibres, such as hair, they can overcome at least one of these drawbacks, with conditioning and remanent effects that can be superior to those of the systems previously used, without, however, impairing at least one of the intensity and homogeneity of the permanent-reshaping, dyeing and bleaching results.

Thus, the condition of the fibres can be improved and the keratin fibres can maintain at least one of their soft feel, their ease of disentangling and their sheen after shampooing several times.

As used herein, the phrase “improvement in the condition of the fibre” means a reduction in the porosity or the alkaline solubility of the fibre and an improvement in at least one cosmetic property, for example, in the smoothness, softness and ease of disentangling and of styling.

This effect can be remanent, i.e., long-lasting.

The porosity is measured by fixing, at 37° C. and at pH 10, for 2 minutes, 2-nitro-para-phenylenediamine at 0.25% in an ethanol/pH 10 buffer mixture (10/90 volume ratio).

The alkaline solubility corresponds to the loss of mass of a sample of 100 mg of keratin fibres under the action of decinormal sodium hydroxide for 30 minutes at 65° C.

One new embodiment relates to a cosmetic composition for treating human keratin fibres, such as hair, comprising, in a cosmetically acceptable medium:

(i) at least one oxidizing agent, and

(ii) at least one aminosilicone comprising at least one aminoethylimino(C₄-C₈)alkyl group; as well as a method of manufacturing this composition.

However, other characteristics, aspects, subjects and advantages of certain embodiments will emerge even more clearly on reading the description and the examples that follow without, however, exhibiting a limiting character.

Aminosilicones

An illustrative at least one aminosilicone has the following formula:

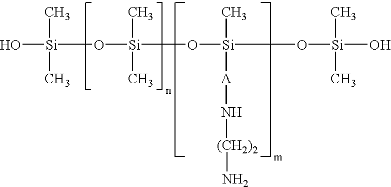

wherein:

A is chosen from linear and branched C₄-C₈ alkylene radicals, for example, C₄ alkylene radicals and

m and n are numbers such that the sum (n+m) can range, for example, from 1 to 2000 and, further for example, from 50 to 150, n can be a number ranging from 0 to 1999, for example, from 49 to 149, and m can be a number ranging from 1 to 2000, for example, from 1 to 10.

The term “alkylene radical” means divalent saturated hydrocarbon-based groups.

The viscosity of the at least one aminosilicone, for example, can be greater than 25 000 mm²/s at 25° C.

For example, this viscosity can range from 30 000 to 200 000 mm²/s at 25° C. and, further for example, from 30 000 to 150 000 mm²/s at 25° C.

The viscosity of the at least one aminosilicone is measured at 25° C. according to standard “ASTM 445 Appendix C.”

The at least one aminosilicone has a weight-average molecular mass, for example, ranging from 2000 to 1 000 000, further for example, from 3500 to 200 000.

The weight-average molecular masses of the at least one aminosilicone is measured by Gel Permeation Chromatography (GPC) at room temperature, as polystyrene equivalents. The columns used are styragel μ columns. The eluent is THF and the flow rate is 1 ml/minute. 200 μl of a solution at 0.5% by weight of silicone in THF is injected. The detection is performed by refractometry and UV-metry.

When the at least one aminosilicone is used, one new embodiment involves using the at least one aminosilicone in the form of an oil-in-water emulsion. The oil-in-water emulsion may comprise at least one surfactant. The at least one surfactant may be of any nature, for example, cationic and/or nonionic.

The silicone particles in the emulsion may have a mean size ranging, for example, from 3 to 500 nanometers, and further for example, from 5 to 300 nanometers, even further for example, from 10 to 275 nanometers and even further for example, from 150 to 275 nanometers. Such particle sizes are measured with a laser granulometer.

An example of the silicone corresponding to this formulation is DC2-8299® from the company Dow Corning.

Another new embodiment uses at least one aminosilicone in the oxidizing composition in an amount ranging from 0.01% to 20% by weight relative to the total weight of the composition. For example, this amount may range from 0.1% to 15% by weight and, further for example, from 0.5% to 10% by weight relative to the total weight of the composition.

Oxidizing Agent

The oxidizing agent can be chosen from hydrogen peroxide and compounds capable of producing hydrogen peroxide by hydrolysis, and mixtures thereof.

For example, the oxidizing agent of the composition can be chosen from aqueous hydrogen peroxide solution, urea peroxide, and persalts such as perborates and persulphates, and mixtures thereof.

Further, for example, the oxidizing agent can be hydrogen peroxide, and even further, for example, the oxidizing agent can be an aqueous hydrogen peroxide solution.

The concentration of hydrogen peroxide may range, for example, from 0.5 to 40 volumes, and further, for example, from 2 to 30 volumes, and the concentration of the compound capable of forming hydrogen peroxide by hydrolysis may range, for example, from 0.1% to 25% by weight relative to the total weight of the oxidizing composition.

The oxidizing compositions may be anhydrous or aqueous.

The oxidizing compositions can be, for example, aqueous, and the pH of the aqueous oxidizing composition may range, for example, from 1 to 13 and further, for example, from 2 to 12.

The oxidizing composition may also, for example, in the case of bleaching, be in the form of two parts to be mixed together at the time of use, one of these two parts comprising alkaline agents and being in solid or liquid form. For hydrogen peroxide, the pH can be, for example, below 7 before mixing.

The pH of the aqueous oxidizing compositions may be obtained and/or adjusted conventionally either by adding basifying agents, alone or as a mixture, such as aqueous ammonia, monoethanolamine, diethanolamine, triethanolamine, isopropanolamine, 1,3-propanediamine, an alkali metal or ammonium carbonate and bicarbonate, an organic carbonate such as guanidine carbonate, and an alkali metal hydroxide, or by adding acidifying agents, alone or as a mixture, such as hydrochloric acid, acetic acid, lactic acid and boric acid.

The oxidizing composition may further comprise additives that are known by one skilled in the art for their use in oxidizing compositions for dyeing the hair by oxidation and for permanently reshaping or bleaching the hair, such as acidifying or basifying agents, preserving agents or sequestering agents.

In one new embodiment, when the oxidizing agent is an aqueous hydrogen peroxide solution, the oxidizing composition comprises at least one stabilizer for the aqueous hydrogen peroxide solution. In the compositions combining aqueous hydrogen peroxide solution and the at least one aminosilicone, for example, advantageous results have been obtained by further including at least one stabilizer chosen from alkali metal and alkaline-earth metal pyrophosphates, alkali metal and alkaline-earth metal stannates, phenacetin and salts of acids and of oxyquinoline, for example, oxyquinoline sulphate. Further, as another example, at least one stannate optionally in combination with at least one pyrophosphate can be used.

In the oxidizing compositions, the concentration of the at least one stabilizer for the aqueous hydrogen peroxide solution may range, for example, from 0.0001% to 5% by weight, and further, for example, from 0.01% to 2% by weight relative to the total weight of the oxidizing compositions.

In the oxidizing compositions with aqueous hydrogen peroxide solution, the concentration ratio of hydrogen peroxide to the at least one stabilizer can range, for example, from 0.05:1 to 1000:1, further, for example, from 0.1:1 to 500:1 and even further, for example, from 1:1 to 200:1. Further, in another new embodiment the concentration ratio of the at least one aminosilicone to the at least one oxidizing agent ranges, for example, from 0.001:1 to 10:1, the amounts of the said silicone and the said oxidizing agent being expressed as active materials (similarly, the amount of hydrogen peroxide for the aqueous hydrogen peroxide solution is expressed as active materials). In another embodiment, the concentration ratio of the at least one aminosilicone to the at least one oxidizing agent may range, for example, from 0.01:1 to 5:1 and even further, for example, from 0.02:1 to 1:1.

Medium

The medium for the composition can be, for example, an aqueous medium comprising water and can, for example, further comprise cosmetically acceptable organic solvents comprising, for example, alcohols such as ethanol, isopropyl alcohol, benzyl alcohol and phenylethyl alcohol, or glycols or glycol ethers such as ethylene glycol monomethyl, monoethyl or monobutyl ether, propylene glycol or its ethers such as propylene glycol monomethyl ether, butylene glycol, dipropylene glycol, and also diethylene glycol alkyl ethers such as diethylene glycol monoethyl ether and monobutyl ether. The organic solvent may then be present in a concentration ranging, for example, from 0.5% to 20%, further, for example, from 2% to 10% by weight relative to the total weight of the composition.

Ingredients

The compositions may also comprise agents for adjusting the rheology such as cellulosic thickeners (hydroxyethylcellulose, hydroxypropylcellulose, carboxymethylcellulose, etc.), guar gum and its derivatives (hydroxypropyl guar, etc.), gums of microbial origin (xanthan gum, scleroglucan gum, etc.), and synthetic thickeners, such as crosslinked homopolymers of acrylic acid and of acrylamidopropanesulphonic acid.

The composition may also comprise at least one ionic or nonionic associative polymer chosen, for example, from the polymers sold under the names Pemulen® TR1 and TR2 by the company Goodrich, Salcare SC 90® by the company Allied Colloids, Aculyn® 22, 28, 33, 44 or 46 by the company Rohm & Haas, and Elfacos® T210 and T212 by the company Akzo, in an amount ranging, for example, from 0.01% to 10% by weight relative to the total weight of the composition.

Further, the composition can also comprise at least one cationic or amphoteric polymer that is well known in the art in the field of dyeing human keratin fibres, in an amount ranging, for example, from 0.01% to 10% by weight, and further, for example, from 0.05% to 5% and even further, for example, from 0.1% to 3% by weight relative to the total weight of the composition.

Cationic Polymers

As used herein, the expression “cationic polymer” means any polymer comprising cationic groups and/or groups which may be ionized into cationic groups.

The cationic polymers which can be used may be chosen from any of those already known by those skilled in the art as improving at least one cosmetic property of the hair, for example, those described in patent application EP-A-337 354 and in French Patent Nos. FR-2 270 846, 2 383 660, 2 598 611, 2 470 596 and 2 519 863.

The cationic polymers may, for example, be chosen from those comprising units comprising at least one amine group chosen from primary, secondary, tertiary and quaternary amine groups, which may either form part of the main polymer chain or may be borne by a side substituent directly attached to the main polymer chain.

The cationic polymers generally have a number-average molecular mass ranging, for example, from 500 to 5×10⁶ and further, for example, from 10³ to 3×10⁶.

Among the cationic polymers which may be mentioned, for example, are polymers of polyamine, polymers of polyamino amide and polymers of polyquaternary ammonium. These polymers are known in the art.

The polymers of polyamine, polymers of polyamino amide and polymers of polyquaternary ammonium are described, for example, in French Patent Nos. 2 505 348 and 2 542 997. Among the said polymers, mention may be made of:

(1) The homopolymers or copolymers derived from acrylic or methacrylic esters or amides and comprising at least one unit chosen from units of formula (I), (II), (III) and (IV) below:

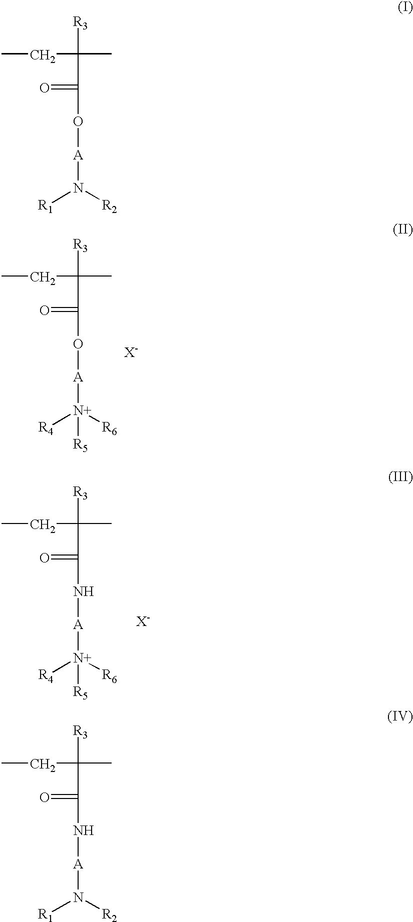

wherein:

R₃, which may be identical or different, is chosen from a hydrogen atom and a CH₃ radical;

A, which may be identical or different, is chosen from linear and branched alkyl groups of 1 to 6 carbon atoms, such as 2 or 3 carbon atoms, and hydroxyalkyl groups of 1 to 4 carbon atoms;

R₄, R₅ and R₆, which may be identical or different, are chosen from alkyl groups comprising from 1 to 18 carbon atoms, for example, alkyl groups comprising from 1 to 6 carbon atoms and benzyl radicals;

R₁ and R₂, which may be identical or different, are chosen from hydrogen and alkyl groups comprising from 1 to 6 carbon atoms, for example, methyl and ethyl groups;

X⁻ is an anion derived from an inorganic or organic acid, such as a methosulphate anion or an anion chosen from halides such as chloride and bromide.

The polymers of family (1) can also comprise at least one unit derived from comonomers, that may be chosen from the family of acrylamides, methacrylamides, diacetoneacrylamides, acrylamides and methacrylamides substituted on the nitrogen with at least one group chosen from lower (C₁-C₄) alkyls, acrylic acids, methacrylic acids, acrylic esters, methacrylic esters, and vinyllactams, such as vinylpyrrolidone, vinylcaprolactam, and vinyl esters.

Thus, among these polymers of family (1), mention may be made of:

the copolymers of acrylamide and of dimethylaminoethyl methacrylate quaternized with dimethyl sulphate or with a dimethyl halide, such as the product sold under the name Hercofloc by the company Hercules,

copolymers of acrylamide and of methacryloyloxyethyltrimethylammonium chloride described, for example, in patent application EP-A-080 976 and sold under the name Bina Quat P 100 by the company Ciba Geigy,

the copolymer of acrylamide and of methacryloyloxyethyltrimethylammonium methosulphate sold under the name Reten by the company Hercules,

quaternized and non-quaternized vinylpyrrolidone/dialkylaminoalkyl acrylate or methacrylate copolymers, such as the products sold under the name “Gafquat” by the company ISP, for example, “Gafquat 734” or “Gafquat 755”, or the products known as “Copolymer 845, 958 and 937.” These polymers are described in detail in French Patent Nos. 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold, for example, under the name Styleze CC 10 by ISP, and

quaternized vinylpyrrolidone/dimethylaminopropylmethacrylamide copolymers such as the product sold under the name “Gafquat HS 100” by the company ISP.

(2) The cellulose ether derivatives comprising quaternary ammonium groups, described in French Patent No. 1 492 597, and, for example, the polymers sold under the names “JR” (JR 400, JR 125 and JR 30M) or “LR” (LR 400, or LR 30M) by the company Union Carbide Corporation. These polymers are also defined in the CTFA dictionary as quaternary ammoniums of hydroxyethylcellulose which has reacted with an epoxide substituted with a trimethylammonium group.

(3) The cationic cellulose derivatives such as cellulose copolymers or cellulose derivatives grafted with a water-soluble monomer of quaternary ammonium, described, for example, in U.S. Pat. No. 4,131,576, such as hydroxyalkylcelluloses, for instance, hydroxymethylcelluloses, hydroxyethylcelluloses, or hydroxypropylcelluloses grafted, for example, with a salt chosen from methacryloyloxyethyltrimethylammonium salts, methacrylamidopropyltrimethylammonium salts and dimethyldiallylammonium salts.

The commercial products corresponding to this definition are, for example, the products sold under the names “Celquat L 200” and “Celquat H 100” by the company National Starch.

(4) The cationic polysaccharides described, for example, in U.S. Pat. Nos. 3,589,578 and 4,031,307, such as guar gums comprising cationic trialkylammonium groups. For example, guar gums modified with a salt (e.g. chloride) of 2,3-epoxypropyltrimethylammonium may be used.

Such products are sold, for example, under the trade names Jaguar C13 S, Jaguar C 15, Jaguar C 17 or Jaguar C162 by the company Meyhall.

(5) Polymers comprising piperazinyl units and divalent alkylene or hydroxyalkylene radicals comprising straight or branched chains, optionally interrupted by at least one atom chosen from oxygen, sulphur and nitrogen atoms or by at least one aromatic or heterocyclic ring, and also at least one of the oxidation and/or quaternization products of these polymers. Such polymers are described, for example, in French Patent Nos. 2 162 025 and 2 280 361.

(6) Water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyidiamine, a bis-alkyl halide or with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine function, they can be quaternized. Such polymers are described, for example, in French Patent Nos. 2 252 840 and 2 368 508.

(7) The polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and, for example, may be chosen from methyl, ethyl and propyl groups. Such polymers are described, for example, in French Patent No. 1 583 363.

Among these derivatives, mention may be made, for example, of the adipic acid/dimethylaminohydroxypropyl/diethylenetriamine polymers sold under the name “Cartaretine F, F4 or F8” by the company Sandoz.

(8) The polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms. The molar ratio between the polyalkylene polyamine and the dicarboxylic acid may range, for example, from 0.8:1 to 1.4:1; the polyamino amide resulting therefrom may be reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Other non-limiting examples of such derivatives include the adipic acid/epoxypropyl/diethylenetriamine copolymers sold, for example, under the name “Hercosett 57” by the company Hercules Inc. or under the name “PD 170” or “Delsette 101” by the company Hercules.

(9) Cyclopolymers of alkyldiallylamine or of dialkyldiallylammonium, such as the homopolymers or copolymers comprising, as main constituent of the chain, at least one of the units corresponding to formula (V) or (VI):

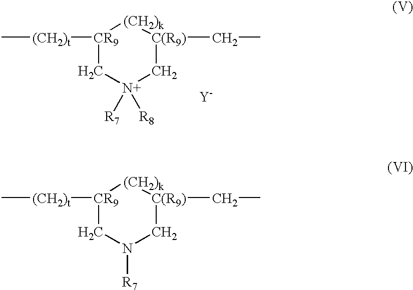

wherein k and t are equal to 0 or 1, the sum k+t being equal to 1;

R₉ is chosen from a hydrogen atom and a methyl radical;

R₇ and R₈, which may be identical or different, are chosen from alkyl groups comprising from 1 to 8 carbon atoms, hydroxyalkyl groups in which the alkyl group, for example, comprises from 1 to 5 carbon atoms, and lower C₁-C₄ amidoalkyl groups, or R₇ and R₈ can be chosen from, together with the nitrogen atom to which they are attached, heterocyclic groups such as piperidyl or morpholinyl; in one new embodiment, R₇ and R₈, which may be identical or different, are chosen from alkyl groups comprising from 1 to 4 carbon atoms; Y⁻ is an anion such as bromide, chloride, acetate, borate, citrate, tartrate, bisulphate, bisulphite, sulphate and phosphate. These polymers are described, for example, in French Patent No. 2 080 759 and in its Certificate of Addition 2 190 406.

Among the polymers defined above, mention may be made, for example, of the dimethyldiallylammonium chloride homopolymer sold under the name “Merquat 100” by the company Calgon (and its homologues of low weight-average molecular mass) and copolymers of diallyldimethylammonium chloride and of acrylamide, sold under the name “Merquat 550.”

(10) The quaternary diammonium polymer comprising repeating units corresponding to the formula (VII):

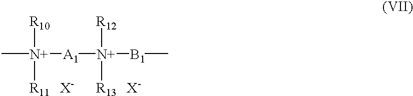

wherein:

R₁₀, R₁₁, R₁₂ and R₁₃, which may be identical or different, are chosen from aliphatic radicals, alicyclic radicals and arylaliphatic radicals comprising from 1 to 20 carbon atoms and from lower hydroxyalkylaliphatic radicals, or R₁₀, R₁₁, R₁₂ and R₁₃, together or separately, constitute, with the nitrogen atoms to which they are attached, heterocycles optionally comprising a second hetero atom other than nitrogen, or R₁₀, R₁₁, R₁₂ and R₁₃ are chosen from linear and branched C₁-C₆ alkyl radicals substituted with at least one group chosen from nitrile, ester, acyl and amide groups and groups of formulae —CO—O—R₁₄-D and —CO—NH—R₁₄-D wherein R₁₄ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen, sulphur atoms and sulphoxide, sulphone, disulphide, amino, alkylamino, hydroxyl, quaternary ammonium, ureido, amide and ester groups, and

X⁻ is an anion chosen from anions derived from mineral and organic acids;

A₁, R₁₀ and R₁₂ may optionally form, with the two nitrogen atoms to which they are attached, a piperazine ring. In addition, if A₁ is a radical chosen from linear and branched, saturated and unsaturated alkylene and hydroxyalkylene radicals, B₁ can also denote a group —(CH₂)ₙ—CO-D-OC—(CH₂)ₙ— wherein n ranges from 1 to 100, such as from 1 to 50.

D is chosen from:

a) a glycol residue of formula: —O-Z-O—, wherein Z is chosen from linear and branched hydrocarbon-based radicals and a group corresponding to one of the following formulae:

—(CH₂—CH₂—O)ₓ—CH₂—CH₂— and

—[CH₂—CH(CH₃)—O]_{y}—CH₂—CH(CH₃)—

wherein x and y, which may be identical or different, are each an integer ranging from 1 to 4, representing a defined and unique degree of polymerization or any number ranging from 1 to 4 representing an average degree of polymerization;

b) a bis-secondary diamine residue such as a piperazine derivative;

c) a bis-primary diamine residue of formula: —NH—Y—NH—, wherein Y is chosen from linear and branched hydrocarbon-based radicals, and the divalent radical —CH₂—CH₂—S—S—CH₂—CH₂—; and

d) a ureylene group of formula: —NH—CO—NH—;

In one embodiment, X⁻ is an anion such as chloride or bromide.

These polymers may have a number-average molecular mass ranging from 1000 to 100 000.

These polymers are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Further, the polymers that comprise repeating units corresponding to formula (VIII) below can be used:

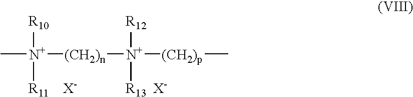

wherein R₁₀, R₁₁, R₁₂ and R₁₃, which may be identical or different, are chosen from alkyl and hydroxyalkyl radicals comprising from 1 to 4 carbon atoms, n and p, which may be identical or different, are integers ranging from 2 to 20, and X⁻ is an anion chosen from anions derived from mineral and organic acids.

(11) Polyquaternary ammonium polymers comprising repeating units of formula (IX):

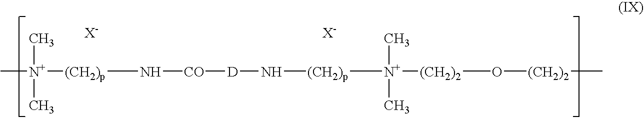

wherein p is an integer ranging from 1 to 6, D may be nothing or may represent a group —(CH₂)ᵣ—CO— in which r may be an integer ranging from 4 to 7, and X⁻ is an anion.

Such polymers may be prepared according to the processes described in U.S. Pat. Nos. 4,157,388, 4,702,906 and 4,719,282. They are described, for example, in patent application EP-A-122 324.

Among these polymers, mention may be made, for example, of “Mirapol A 15”, “Mirapol AD1”, “Mirapol AZ1” and “Mirapol 175” sold by the company Miranol.

(12) Quaternary polymers of vinylpyrrolidone and of vinylimidazole, such as the products sold under the names Luviquat FC 905, FC 550 and FC 370 by the company BASF.

(13) Polyamines such as Polyquart H sold by Henkel, under the reference name “Polyethylene glycol (15) tallow polyamine” in the CTFA dictionary.

(14) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide. A crosslinked acrylamide/methacryloyloxyethyltrimethylammonium chloride copolymer (20/80 by weight) in the form of a dispersion comprising 50% by weight of the said copolymer in mineral oil can, for example, be used. This dispersion is sold under the name “Salcare® SC 92” by the company Allied Colloids. In another embodiment, a crosslinked methacryloyloxyethyltrimethylammonium chloride homopolymer comprising about 50% by weight of the homopolymer in mineral oil or in a liquid ester can also be used. These dispersions are sold under the names “Salcare® SC 95” and “Salcare® SC 96” by the company Allied Colloids.

Other cationic polymers are chosen from polyalkyleneimines, for example, polyethyleneimines, polymers comprising vinylpyridine and vinylpyridinium units, condensates of polyamines and of epichlorohydrin, quaternary polyureylenes and chitin derivatives.

Among all the cationic polymers that may be used, non-limiting examples include the polymers of families (1), (9), (10), (11) and (14) and, in one new embodiment, polymers comprising repeating units chosen from units of formulae (W) and (U) below can be used:

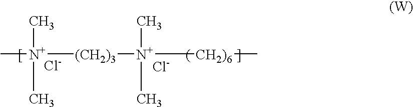

and, for example, those polymers comprising repeating units of formula (W) whose weight-average molar mass, determined by gel permeation chromatography, ranges from 9500 to 9900;

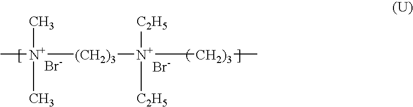

and, for example, those polymers comprising repeating units of formula (U) whose weight-average molar mass, determined by gel permeation chromatography, is about 1200.

The concentration of the cationic polymer in the composition may range, for example, from 0.01% to 10% by weight relative to the total weight of the composition, and further, for example from 0.05% to 5% by weight and even further, for example, from 0.1% to 3% by weight relative to the total weight of the composition.

Amphoteric Polymers

The amphoteric polymers, may be chosen from polymers comprising units K and M randomly distributed in the polymer chain, in which K is a unit derived from a monomer comprising at least one basic nitrogen atom and M is a unit derived from an acidic monomer comprising at least one carboxylic or sulphonic group, or K and M may be chosen from groups derived from zwitterionic carboxybetaine or sulphobetaine monomers.

K and M may also be chosen from a cationic polymer chain comprising at least one group chosen from primary, secondary, tertiary and quaternary amine groups, in which at least one of the amine groups bears a carboxylic or sulphonic group linked via a hydrocarbon-based radical, or K and M can form part of a chain of a polymer comprising an α,β-dicarboxylic ethylene unit in which one of the carboxylic groups has been made to react with a polyamine comprising at least one amine chosen from primary and secondary amine groups.

The amphoteric polymers corresponding to the above definition, for example, are chosen from the following polymers:

(1) polymers resulting from the copolymerization of at least one monomer derived from a vinyl compound bearing a carboxylic group such as acrylic acid, methacrylic acid, maleic acid, α-chloroacrylic acid, and at least one basic monomer derived from a substituted vinyl compound comprising at least one basic atom, such as dialkylaminoalkyl methacrylate and acrylate, dialkylaminoalkylmethacrylamide and -acrylamide. Such compounds are described, for example, in U.S. Pat. No. 3,836,537.

Mention may also be made of the sodium acrylate/acrylamidopropyltrimethylammonium chloride copolymer sold under the name Polyquart KE 3033 by the company Henkel.

The vinyl compound may also be a dialkyldiallylammonium salt such as dimethyldiallylammonium chloride. The copolymers of acrylic acid and of the latter monomer are sold under the names Merquat 280, Merquat 295 and Merquat Plus 3330 by the company Calgon.

(2) polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one new embodiment, the N-substituted acrylamides or methacrylamides are, for example, groups in which the alkyl radicals comprise from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acids, methacrylic acids, crotonic acids, itaconic acids, maleic acids and fumaric acids and alkyl monoesters, comprising 1 to 4 carbon atoms, of maleic or fumaric acids or anhydrides.

The basic comonomers are chosen, for example, from aminoethyl, butylaminoethyl, N,N′-dimethylaminoethyl and N-tert-butylaminoethyl methacrylates.

The copolymers having the CTFA (4th edition, 1991) name octylacrylamide/acrylates/butylaminoethyl methacrylate copolymer such as the products sold under the name Amphomer or Lovocryl 47 by the company National Starch can, for example, be used.

(3) crosslinked and alkylated polyamino amides partially or totally derived from polyamino amides of general formula:

[CO—R₁₉—CO—Z] (X)

wherein R₁₉ is chosen from a divalent radical derived from a saturated dicarboxylic acid, mono- and dicarboxylic aliphatic acids comprising an ethylenic double bond, an ester of a lower alkanol, comprising 1 to 6 carbon atoms, of these acids and a radical derived from the addition of any one of the acids to amines chosen from bis(primary) and bis(secondary) amines, and Z is chosen from bis(primary), mono- and bis(secondary) polyalkylene-polyamine radicals and, for example, Z represents:

a) in proportions ranging from 60 to 100 mol %, the radical of formula (XI)

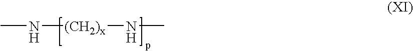

wherein x=2 and p=2 or 3, or x=3 and p=2,

this radical being derived from a compound chosen from diethylenetriamine, triethylenetetraamine and dipropylenetriamine;

b) in proportions ranging from 0 to 40 mol %, the radical (XI) above in which x=2 and p=1 and which is derived from a compound chosen from ethylenediamine, and piperazine:

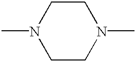

c) in proportions ranging from 0 to 20 mol %, the —NH—(CH₂)₆—NH— radical, which is derived from hexamethylenediamine, these polyamino amines can be crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one embodiment, the saturated carboxylic acids are chosen from acids comprising 6 to 10 carbon atoms, such as adipic acid, 2,2,4-trimethyladipic acid and 2,4,4-trimethyladipic acid, terephthalic acid and acids comprising an ethylenic double bond such as, for example, acrylic acid, methacrylic acid and itaconic acid.

The alkane sultones used in the alkylation are chosen, for example, from propane sultone and butane sultone, and the salts of the alkylating agents can be chosen, for example, from the sodium and potassium salts.

(4) polymers comprising zwitterionic units of formula (XII):

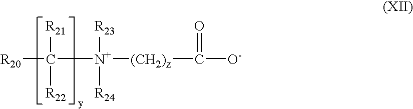

wherein R₂₀ is chosen from polymerizable unsaturated groups such as acrylate, methacrylate, acrylamide and methacrylamide groups, y and z, which may be identical or different, are chosen from integers ranging from 1 to 3, R₂, and R₂₂, which may be identical or different, are chosen from a hydrogen atom, methyl, ethyl and propyl groups, R₂₃ and R₂₄, which may be identical or different, are chosen from a hydrogen atom and alkyl radicals such that the sum of the carbon atoms in R₂₃ and R₂₄ does not exceed 10.

The polymers comprising such units can also comprise units derived from non-zwitterionic monomers such as monomers chosen from dimethyl and diethylaminoethyl acrylates, methacrylates, alkyl acrylates, methacrylates, acrylamides, methacrylamides, and vinyl acetate.

By way of example, mention may be made of the copolymer of methyl methacrylate/methyl dimethylcarboxymethylammonioethylmethacrylate such as the product sold under the name Diaformer Z301 by the company Sandoz.

(5) polymers derived from chitosan comprising monomer units corresponding to formulae (XIII), (XIV) and (XV) below:

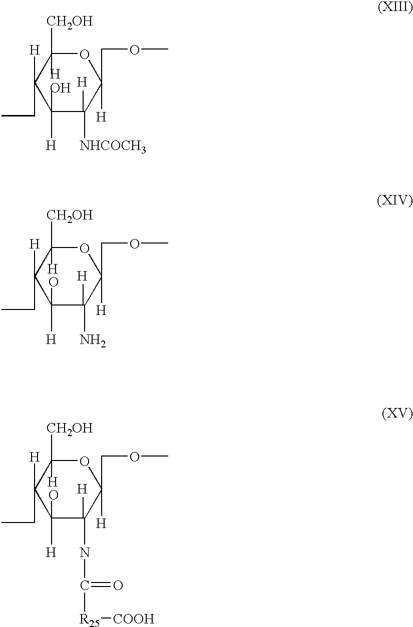

the unit (XIII) being present in proportions ranging from 0 to 30%, the unit (XIV) in proportions ranging from 5% to 50% and the unit (XV) in proportions ranging from 30% to 90%, and wherein in unit (XV), R₂₅ is a radical of formula:

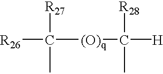

wherein q denotes 0 or 1;

if q=0, R₂₆, R₂₇ and R₂₈, which may be identical or different, are each chosen from a hydrogen atom, methyl, hydroxyl, acetoxy and amino residues, and monoalkylamine residues and dialkylamine residues, which are optionally interrupted by at least one nitrogen atom and/or optionally substituted with at least one amine, hydroxyl, carboxyl, alkylthio and sulphonic groups, and alkylthio residues in which the alkyl group bears an amino residue, at least one of the radicals R₂₆, R₂₇ and R₂₈ being, in this case, a hydrogen atom;

or, if q=1, R₂₆, R₂₇ and R₂₈, which may be identical or different, are each chosen from a hydrogen atom, and salts formed by these compounds with bases or acids.

(6) polymers derived from the N-carboxyalkylation of chitosan, such as N-carboxymethylchitosan or N-carboxybutylchitosan sold under the name “Evalsan” by the company Jan Dekker.

(7) polymers corresponding to the general formula (XVI) as described, for example, in French Patent No. 1 400 366:

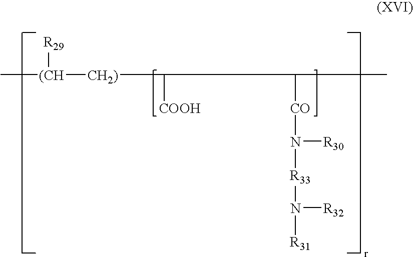

wherein R₂₉ is chosen from a hydrogen atom, and CH₃O, CH₃CH₂O and phenyl radicals, R₃₀ is chosen from a hydrogen atom and lower alkyl radicals such as methyl and ethyl, R₃₁ is chosen from a hydrogen atom and lower alkyl radicals such as methyl and ethyl, R₃₂ is chosen from lower alkyl radicals such as methyl and ethyl and radicals corresponding to the formula: —R₃₃—N(R₃₁)₂, wherein R₃₃ is chosen from —CH₂—CH₂—, —CH₂—CH₂—CH₂— and —CH₂—CH(CH₃)— groups, and R₃₁ is chosen from a hydrogen atom and lower alkyl radicals such as methyl and ethyl, and also the higher homologues of these radicals comprising up to 6 carbon atoms,

r is chosen such that the number-average molecular weight of said polymer ranges from 500 to 6 000 000, such as from 1000 to 1 000 000.

(8) amphoteric polymers of the type -D-X-D-X— chosen from:

a) polymers obtained by the action of chloroacetic acid or sodium chloroacetate on compounds comprising at least one unit of formula:

-D-X-D-X-D- (XVII)

wherein D is a radical

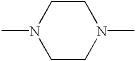

and X is chosen from the symbols E and E′, wherein E and E′, which may be identical or different, are chosen from divalent alkylene radicals comprising at least one chain chosen from straight and branched chains comprising up to 7 carbon atoms in the main chain, wherein said divalent alkylene radicals are optionally substituted with at least one hydroxyl group. E or E′ can additionally comprise at least one atom chosen from oxygen, nitrogen and sulphur atoms, and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine, hydroxyl, benzylamine, amine oxide, quaternary ammonium, amide, imide, alcohol, ester and urethane groups;

b) polymers of formula:

-D-X-D-X— (XVIII)

wherein D is a radical

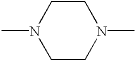

and X is chosen from the symbol E and E′ and wherein at least one X is chosen from E′; E having the meaning given above and E′ is a divalent radical chosen from divalent alkylene radicals comprising at least one chain chosen from straight and branched chains comprising up to 7 carbon atoms in the main chain, wherein said divalent alkylene radicals are optionally substituted with at least one hydroxyl radical and comprise at least one nitrogen atom chain substituted with an alkyl chain, which is optionally interrupted by an oxygen atom, wherein said alkyl chain comprises at least one functional group chosen from carboxyl functional groups and hydroxyl functional groups which are betainized by reaction with a reactant chosen from chloroacetic acid and sodium chloroacetate.

(9) (C₁-C₅)alkyl vinyl ether/maleic anhydride copolymers partially modified by semiamidation with a N,N-dialkylaminoalkylamine such as N,N-dimethylamino propylamine or by semiesterification with a N,N-dialkanolamine. These copolymers can also comprise other vinyl comonomers such as vinylcaprolactam.

In one new embodiment, the amphoteric polymers are those of family (1).

The concentration of the amphoteric polymer may be ranging, for example, from 0.01% to 10% by weight, such as from 0.05% to 5% by weight and further such as from 0.1% to 3% by weight relative to the total weight of the composition.

In another new embodiment, the composition can further comprise at least one surfactant.

The at least one surfactant may be chosen from anionic, amphoteric, nonionic, zwitterionic and cationic surfactants.

The at least one surfactant can be, for example, chosen from:

(i) Anionic Surfactants:

By way of example of anionic surfactants which can be used, alone or as mixtures, mention may be made, for example, of salts such as alkali metal salts, for example, sodium salts, ammonium salts, amine salts, amino alcohol salts or magnesium salts of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄)alkyl sulphosuccinates, (C₆-C₂₄)alkyl ether sulphosuccinates, (C₆-C₂₄)alkylamide sulphosuccinates; (C₆-C₂₄)alkyl sulphoacetates; (C₆-C₂₄)acyl sarcosinates; and (C₆-C₂₄)acyl glutamates. It is also possible to use (C₆-C₂₄)alkylpolyglycoside carboxylic esters such as alkylglucoside citrates, alkylpolyglycoside tartrates and alkylpolyglycoside sulphosuccinates, alkylsulphosuccinamates; acyl isethionates and N-acyl taurates. The alkyl or acyl radical of all of these different compounds comprises, for example, from 12 to 20 carbon atoms and the aryl radical is chosen, for example, from phenyl and benzyl groups. Among the anionic surfactants which can be used, mention may also be made of fatty acid salts such as oleic, ricinoleic, palmitic and stearic acid salts, coconut oil acid or hydrogenated coconut oil acid and their salts; acyl lactylates, wherein the acyl radical comprises from 8 to 20 carbon atoms. It is also possible to use alkyl D-galactoside uronic acids and their salts, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, for example, those comprising from 2 to 50 alkylene oxide groups, such as ethylene oxide groups, and mixtures thereof.

(ii) Nonionic Surfactants:

The nonionic surfactants are compounds that are well known (see, for example, in this respect “Handbook of Surfactants” by M. R. Porter, published by Blackie & Son (Glasgow and London), 1991, pp. 116-178). They can be chosen, for example, from polyethoxylated and polypropoxylated alkylphenols, alpha-diols and alcohols comprising a fatty chain comprising, for example, 8 to 18 carbon atoms, it being possible for the number of ethylene oxide or propylene oxide groups to range, for example, from 2 to 50. Mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average from 1 to 5, such as 1.5 to 4, glycerol groups; polyethoxylated fatty amines such as those comprising from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, and amine oxides such as (C₁₀-C₁₄)alkylamine oxides or N-acylaminopropylmorpholine oxides.

(iii) Amphoteric or Zwitterionic Surfactants:

The amphoteric or zwitterionic surfactants can be chosen, for example, from aliphatic secondary and tertiary amine derivatives wherein the aliphatic radical is chosen from linear and branched chains comprising 8 to 18 carbon atoms and comprising at least one water-solubilizing anionic group (for example carboxylate, sulphonate, sulphate, phosphate or phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, mention may be made of the products sold under the name Miranol, as described in U.S. Pat. Nos. 2,528,378 and 2,781,354 and classified in the CTFA dictionary, 3rd edition, 1982, under the names amphocarboxyglycinates and amphocarboxypropionates, with the respective structures of:

R₂—CONHCH₂CH₂—N⁺(R₃)(R₄)(CH₂COO⁻)

wherein: R₂ is chosen from alkyl radicals derived from an acid R₂—COOH present in hydrolysed coconut oil, and heptyl, nonyl and undecyl radicals, R₃ is a beta-hydroxyethyl group and R₄ is a carboxymethyl group;

and of

R₂′-CONHCH₂CH₂—N(B)(C)

wherein:

B represents —CH₂CH₂OX′, C represents —(CH₂)_{z}—Y′, with z=1 or 2,

X′ is chosen from the —CH₂CH₂—COOH group and a hydrogen atom,

Y′ is chosen from the —COOH and the —CH₂—CHOH—SO₃H radicals,

R′₂ is chosen from alkyl radicals of an acid R′₂—COOH present in coconut oil or in hydrolysed linseed oil, alkyl radicals, such as C₇, C₉, C₁₁ and C₁₃ alkyl radicals, a C₁₇ alkyl radical and its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphopropionate, disodium caprylamphodipropionate, disodium caprylamphodipropionate, lauroamphodipropionic acid and cocoamphodipropionic acid.

By way of example, mention may be made of the cocoamphodiacetate sold under the trade name Miranol® C2M concentrate by the company Rhodia Chimie.

(iv) Cationic Surfactants:

The cationic surfactants, may be chosen, for example, from: salts of optionally polyoxyalkylenated primary, secondary and tertiary fatty amines; quaternary ammonium salts such as tetraalkylammonium, alkylamidoalkyltrialkylammonium, trialkylbenzylammonium, trialkylhydroxyalkylammonium and alkylpyridinium chlorides and bromides; imidazoline derivatives; and amine oxides of cationic nature.

The amount of the surfactants present in the composition can range, for example, from 0.01% to 40% by weight such as from 0.5% to 30% by weight relative to the total weight of the composition.

Needless to say, a person skilled in the art will take care to select the optional additional compound(s) mentioned above, such that at least one advantageous property intrinsically associated with the oxidizing composition are not, or are not substantially, adversely affected by the envisaged addition(s).

Another new embodiment is a process for the oxidation dyeing of human keratin fibres, such as hair, using a dye composition comprising, in a cosmetically acceptable medium, at least one oxidation dye and an oxidizing composition as defined above.

According to this process, at least one dye composition as defined above is applied to the fibres, the colour being developed at acidic, neutral or alkaline pH using an oxidizing composition, which is applied simultaneously or sequentially, with or without intermediate rinsing.

According to one new embodiment of the dyeing process, the dye composition described above is mixed, at the time of use, with an oxidizing composition. The mixture obtained is then applied to the keratin fibres and is left to act for about 3 to 50 minutes, such as about 5 to 30 minutes, after which the fibres are rinsed, washed with shampoo, rinsed again and dried.

Another new embodiment is a process for permanently reshaping human keratin fibres, such as hair, using the composition defined above as an oxidizing composition.

Initially, this process comprises applying to the hair a reducing composition. This application is performed lock by lock or globally.

The reducing composition comprises at least one reducing agent, which may be chosen, for example, from thioglycolic acid, cysteine, cysteamine, glyceryl thioglycolate, thiolactic acid, and salts of thiolactic and thioglycolic acid.

The usual placement of the hair under tension in a shape corresponding to the final shape desired for the hair (for example curls) may be carried out by any means, such as mechanical means, that is suitable and known by those skilled in the art for keeping hair under tension, such as rollers, curlers or the like.

The hair may also be shaped without the aid of external means, simply with the fingers.

Before proceeding to the following optional rinsing, the head of hair onto which the reducing composition has been applied should conventionally be left to stand for a few minutes, generally ranging from five minutes to one hour, for example, from 10 to 30 minutes, so as to give the reducing agent enough time to act correctly on the hair. This waiting phase is, for example, performed at a temperature ranging from 35° C. to 45° C., while also, for example, protecting the hair with a hood.

In the second optional rinsing of the process (rinsing (ii)), the hair impregnated with the reducing composition is then rinsed thoroughly with an aqueous composition.

Next, in fixing (iii), the oxidizing composition is applied to the hair thus rinsed, with the aim of fixing the new shape given to the hair.

As in the case of applying the reducing composition, the head of hair onto which the oxidizing composition has been applied is then conventionally left in a standing or waiting phase that lasts a few minutes, generally ranging from 3 to 30 minutes, for example, from 5 to 15 minutes.

If the hair was held under tension by external means, these means (rollers, curlers or the like) may be removed from the hair before or after fixing.

Finally, in optional rinsing (iv), the hair impregnated with the oxidizing composition is rinsed thoroughly, generally with water.

A head of hair that is easy to disentangle and soft can finally be obtained. The hair can be wavy.

The oxidizing composition may also be used in a process for bleaching human keratin fibres, such as hair.

The bleaching process comprises applying an oxidizing composition to the keratin fibres, this composition comprising, for example, aqueous hydrogen peroxide solution in alkaline medium after extemporaneous mixing. Conventionally, in the bleaching process, the keratin fibres are rinsed.

The examples that follow illustrate new embodiments without being limiting in nature.

### EXAMPLE 1

The ready-to-use aqueous bleaching composition below was prepared (amounts expressed as grams of active material):

| | | |
|---|---|---|
| 200 volumes hydrogen peroxide | | 12 |
| Stabilizer | | qs |
| Silicone: DC2-8299 ® from the company Dow Corning | | 2 |
| pH agent | qs | pH 4.7 |
| Water | qs | 100 |

The above bleaching composition was applied to natural hair and left for 45 minutes under a hood, and then rinsed thoroughly with water. A uniform lightening of the head of hair was obtained, with the hair being soft, light, shiny and easy to disentangle.

### EXAMPLE 2

Permanent-Reshaping Composition:

The reducing composition below was prepared (amounts expressed as grams of active material):

| | | |
|---|---|---|
| Thioglycolic acid | | 9.2 |
| Arginine | | 15 |
| Aqueous ammonia containing 20% NH₃ | | 1.86 |
| Ammonium carbonate | | 4.5 |
| Cocoylamidopropylbetaine/glyceryl monolaurate (25/5) as an | | 1.3 |
| aqueous 30% solution | | |
| Peptizer | | 0.8 |
| Isostearyl alcohol (Tego Alkanol 66 sold by the company | | 12 |
| Goldschmidt) | | |
| Sequestering agent | | 0.4 |
| Fragrance | | 0.4 |
| Demineralized water | qs | 100 |

This reducing composition was applied to a lock of wet hair, rolled up beforehand on a curler 9 mm in diameter.

After leaving the composition to act for ten minutes, the lock was rinsed thoroughly with water.

The oxidizing composition below was then applied:

Oxidizing Composition:

(amounts expressed as grams of active material):

| | | |
|---|---|---|
| Silicone DC2-8299 ® from the company Dow Corning | | 2 |
| Aqueous hydrogen peroxide solution | | 8 volumes |
| Tetrasodium pyrophosphate (0.02 g) and sodium | | |
| stannate (0.04 g) | | |
| Sequestering agent: pentasodium pentaacetate | | 0.06 |
| Demineralized water | qs | 100 |

After leaving the composition to act for ten minutes, the lock was rinsed thoroughly with water. The hair was then unrolled from the roller and dried.

The lock was wavy, with a very good cosmetic condition.

What is claimed is:

1. A cosmetic composition for treating human keratin fibres comprising, in a cosmetically acceptable medium:
(i) at least one oxidizing agent, and
(ii) at least one aminosilicone comprising at least one aminoethylimino(C₄-C₈)alkyl group of the formula below: 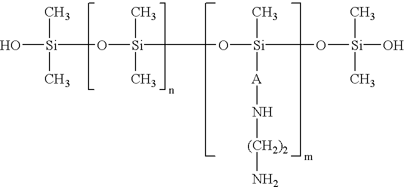
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000.

2. The composition according to claim 1, wherein the human keratin fibres are hair.

3. The composition according to claim 1, wherein the sum (n+m) ranges from 50 to 150.

4. The composition according to claim 1, wherein n is a number ranging from 49 to 149.

5. The composition according to claim 1, wherein m is a number ranging from 1 to 10.

6. The composition according to claim 1, wherein A is chosen from linear and branched C₄ alkylene radicals.

7. The composition according to claim 1, wherein the viscosity of the at least one aminosilicone is greater than 25 000 mm²/s at 25° C.

8. The composition according to claim 7, wherein the viscosity of the at least one aminosilicone ranges from 30 000 to 200 000 mm²/s at 25° C.

9. The composition according to claim 8, wherein the viscosity of the at least one aminosilicone ranges from 30 000 to 150 000 mm²/s at 25° C.

10. The composition according to claim 1, wherein the at least one aminosilicone has a weight-average molecular mass ranging from 2000 to 1 000 000.

11. The composition according to claim 10, wherein the at least one aminosilicone has a weight-average molecular mass ranging from 3500 to 200 000.

12. The composition according to claim 1, wherein the at least one aminosilicone is in the form of an oil-in-water emulsion comprising at least one surfactant.

13. The composition according to claim 12, wherein the oil-in-water emulsion comprises at least one surfactant chosen from cationic and nonionic surfactants.

14. The composition according to claim 12, wherein the particle size of said at least one aminosilicone ranges from 3 to 500 nanometers.

15. The composition according to claim 14, wherein the particle size of said at least one aminosilicone ranges from 5 to 300 nanometers.

16. The composition according to claim 15, wherein the particle size of said at least one aminosilicone ranges from 10 to 275 nanometers.

17. The composition according to claim 16, wherein the particle size of said at least one aminosilicone ranges from 150 to 275 nanometers.

18. The composition according to claim 1, wherein the at least one aminosilicone is present in an amount ranging from 0.01% to 20% by weight relative to the total weight of the composition.

19. The composition according to claim 18, wherein the at least one aminosilicone is present in an amount ranging from 0.1% to 15% by weight relative to the total weight of the composition.

20. The composition according to claim 19, wherein the at least one aminosilicone is present in an amount ranging from 0.5% to 10% by weight relative to the total weight of the composition.

21. The composition according to claim 1, wherein the at least one oxidizing agent is chosen from hydrogen peroxide, urea peroxide, perborates and persulphates.

22. The composition according to claim 21, wherein the at least one oxidizing agent is hydrogen peroxide.

23. The composition according to claim 22, wherein the concentration of the hydrogen peroxide ranges from 0.5 to 40 volumes.

24. The composition according to claim 23, wherein the concentration of the hydrogen peroxide ranges from 2 to 30 volumes.

25. The composition according to claim 22, wherein the at least one oxidizing agent is an aqueous hydrogen peroxide solution.

26. The composition according to claim 25, wherein the aqueous hydrogen peroxide solution comprises at least one stabilizer.

27. The composition according to claim 26, wherein the at least one stabilizer is chosen from alkali metal and alkaline-earth metal pyrophosphates, alkali metal and alkaline-earth metal stannates, phenacetin, and salts of acids and of oxyquinoline.

28. The composition according to claim 26, wherein the at least one stabilizer is chosen from stannates and pyrophosphates.

29. The composition according to claim 26, wherein the concentration of the at least one stabilizer ranges from 0.0001% to 5% by weight relative to the total weight of the composition.

30. The composition according to claim 29, wherein the concentration of the at least one stabilizer ranges from 0.01% to 2% by weight relative to the total weight of the composition.

31. The composition according to claim 26, wherein the concentration ratio of the hydrogen peroxide to the at least one stabilizer ranges from 0.05:1 to 1000:1.

32. The composition according to claim 31, wherein the concentration ratio of the hydrogen peroxide to the at least one stabilizer ranges from 0.1:1 to 500:1.

33. The composition according to claim 32, wherein the concentration ratio of the hydrogen peroxide to the at least one stabilizer ranges from 1:1 to 200:1.

34. The composition according to claim 26, wherein the concentration ratio of the at least one aminosilicone to the at least one stabilizer ranges from 0.05:1 to 1000:1.

35. The composition according to claim 34, wherein the concentration ratio of the at least one aminosilicone to the at least one stabilizer ranges from 0.1:1 to 500:1.

36. The composition according to claim 35, wherein the concentration ratio of the at least one aminosilicone to the at least one stabilizer ranges from 1:1 to 200:1.

37. The composition according to claim 1, wherein the concentration ratio of the at least one aminosilicone to the at least one oxidizing agent ranges from 0.001:1 to 10:1.

38. The composition according to claim 37, wherein the concentration ratio of the at least one aminosilicone to the at least one oxidizing agent ranges from 0.01:1 to 5:1.

39. The composition according to claim 38, wherein the concentration ratio of the at least one aminosilicone to the at least one oxidizing agent ranges from 0.02:1 to 1:1.

40. The composition according to claim 1, wherein the concentration of the at least one oxidizing agent ranges from 0.1% to 25% by weight relative to the total weight of the composition.

41. The composition according to claim 1, wherein the composition is aqueous.

42. The composition according to claim 41, wherein the pH of the composition ranges from 1 to 13.

43. The composition according to claim 42, wherein the pH of the composition ranges from 2 to 12.

44. A process for oxidation dyeing of human keratin fibres comprising applying to the human keratin fibre a dye composition comprising, in a cosmetically acceptable medium, at least one oxidation dye and an oxidizing composition comprising, in a cosmetically acceptable medium,
(i) at least one oxidizing agent, and
(ii) at least one aminosilicone comprising at least one aminoethylimino(C₄-C₈)alkyl group of the formula below: 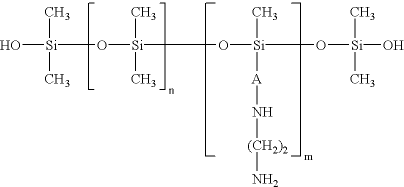
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000.

45. The process according to claim 44, wherein said human keratin fibres are hair.

46. The process according to claim 44, wherein before applying to the human keratin fibres, the dye composition and the oxidizing composition are mixed at the time of use.

47. The process according to claim 46, further comprising:
leaving the mixture of the dye composition and the oxidizing composition on said human keratin fibres for approximately 3 to 50 minutes, and then
rinsing said human keratin fibres, washing said human keratin fibres with shampoo, and further rinsing said human keratin fibres and
drying said human keratin fibres.

48. The process according to claim 47, wherein said leaving the mixture of the dye composition and the oxidizing composition on said human keratin fibres is for approximately 5 to 30 minutes.

49. The process according to claim 44, wherein the dye composition and the oxidizing composition are applied sequentially to the human keratin fibres.

50. The process according to claim 49, further comprising rinsing the human keratin fibres between applying to the human keratin fibres the dye composition and applying to the human keratin fibres the oxidizing composition.

51. A process for treating human keratin fibres to permanently reshape said human keratin fibres comprising:
(A) applying to said human keratin fibres a reducing composition, wherein said human keratin fibres is placed under mechanical tension during or after said application,
(B) optionally rinsing said human keratin fibres,
(C) applying to said human keratin fibres an oxidizing composition comprising, in a cosmetically acceptable medium,
(i) at least one oxidizing agent, and
(ii) at least one aminosilicone comprising at least one aminoethylimino(C₄-C₈)alkyl group of the formula below: 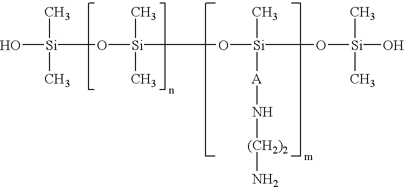
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000, and
(D) optionally rinsing said human keratin fibres.

52. A process according to claim 51, wherein said human keratin fibres are hair.

53. The process according to claim 52, wherein the permanently reshaped hair is in the form of permanent-waved hair.

54. A process for bleaching human keratin fibres comprising:
(A) applying to said human keratin fibres an oxidizing composition comprising, in a cosmetically acceptable medium,
(i) at least one oxidizing agent, and
(ii) at least one aminosilicone comprising at least one aminoethylimino(C₄-C₈)alkyl group of the formula below: 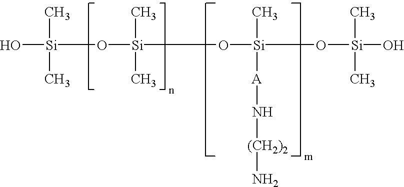
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000, and
(B) rinsing said human keratin fibres.

55. The process according to claim 54, wherein said human keratin fibres are hair.

56. A composition for improving the cosmetic condition of a human keratin fibre comprising, in a cosmetically acceptable medium, at least one aminosilicone comprising at least one aminoethylimino(C₄-C₈)alkyl group of the formula below: 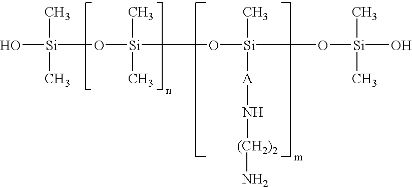
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000,
wherein the composition comprises at least one oxidizing agent, and the at least one aminosilicone is effective in improving the cosmetic condition of the human keratin fibre.

57. A process for improving the cosmetic condition of human keratin fibres comprising applying to the human keratin fibres a cosmetic composition comprising, in a cosmetically acceptable medium, at least one aminosilicone comprising at least one aminoethylimino(C₄-C₈)alkyl group of the formula below: 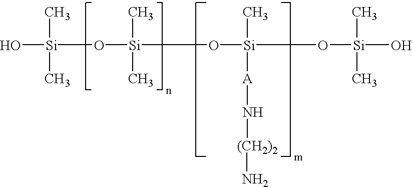
wherein:
A is chosen from linear and branched C₄-C₈ alkylene radicals;
m and n are numbers such that the sum (n+m) ranges from 1 to 2000,
n is a number ranging from 0 to 1999, and
m is a number ranging from 1 to 2000;
wherein the composition comprises at least one oxidizing agent.
